(19) United States
(12) Patent Application Publication
(10) Pub. No.: US 2007/0148129 A1
(43) Pub. Date: Jun. 28, 2007

(54) Therapeutic agents and uses therefor

(75) Inventors: Ken Shortman, Carlton North (AU); Meredith O'Keeffe, West Brunswick (AU); Ben Fancke, Ashburton (AU); Len Harrison, St. Kilda West (AU); Ray Steptoe, Ashgrove (AU); David Vremec, Ferntree Gully (AU)

Correspondence Address: SCULLY, SCOTT, MURPHY & PRESSER, P.C., 400 GARDEN CITY PLAZA, SUITE 300, GARDEN CITY, NY, 11530, US

(73) Assignee: THE WALTER AND ELIZA HALL INSTIUTE OF MEDICAL RESEARCH, Victoria Australia (AU)

(21) Appl. No.: 10/584,180

(22) PCT Filed: Dec. 23, 2004
(86) PCT No.: PCT/AU04/01840

§ 371 Date: Oct. 11, 2006

(30) Foreign Application Priority Data
Dec. 24, 2003 (AU) 2003907195

(51) Int. Cl. A61K 38/19; A61K 39/00

(52) U.S. Cl. 424/85.1; 424/185.1

(57) ABSTRACT

The present invention relates generally to therapeutic agents and methods which enhance or otherwise maintain a state of immune tolerance in a subject. The present invention further provides agents and methods for preventing or at least delaying onset of an autoimmune disease such as but not limited to autoimmune diabetes. Furthermore, the agents and methods of the present invention are useful in enhancing the effectiveness of vaccine regimes such as against cancer cells or pathogenic organisms and viruses or for generally enhancing the immune responsiveness against such entities. The present invention further enables the prevention of pathogenic agent-induced autoimmune conditions.

### BACKGROUND OF THE INVENTION

1. Field of the Invention

The present invention relates generally to therapeutic agents and methods which enhance or otherwise maintain a state of immune tolerance in a subject. The present invention further provides agents and methods for preventing or at least delaying onset of an autoimmune disease such as but not limited to autoimmune diabetes. Furthermore, the agents and methods of the present invention are useful in enhancing the effectiveness of vaccine regimes such as against cancer cells or pathogenic organisms and viruses or for generally enhancing the immune responsiveness against such entities. The present invention further enables the prevention of pathogenic agent-induced autoimmune conditions.

2. Description of the Prior Art

Bibliographic details of references in the subject specification are also listed at the end of the specification.

Reference to any prior art in this specification is not, and should not be taken as, an acknowledgment or any form of suggestion that that prior art forms part of the common general knowledge in any country.

The immune defense system represents a delicate balance between effective responses to invading microorganisms and the avoidance of autoimmune responses to the body's own tissues. There are a series of regulatory control systems which normally prevent or limit autoimmunity, although these sometimes fail. These control systems include “central tolerance” which is the elimination of self-reactive cells within the thymus before they enter the peripheral immune system. This is backed up by several peripheral control mechanisms, which involve the elimination or activation of self-reactive cells and the generation of “regulatory T cells” which dampen down or prevent autoimmune responses.

Dendritic cells (DC) represent a system of antigen-presenting cells which are needed to initiate immune responses by T lymphocytes. It has become clear that as well as initiating immune responses, DC have a major role in regulating immunity (Steinman et al Ann N Y Acad Sci., 987:15-25, 2003, Shortman and Liu, Nat Rev Immunol, 2:153-163, 2002, Belz et al Immunol Cell Biol., 80:463-468, 2002, Matzinger, Annu Rev Immunol., 12:991-1045, 1994). The DC in the thymus play a major role in eliminating developing self-reactive T cells. In the periphery DC can dictate the type of immune responses obtained (eg. Th1 versus Th2). More recent evidence shows DC in peripheral lymphoid organs can play a major role in maintaining self-tolerance. A current general view is that DC in the quiescent or immature state can present self-antigens and induce tolerance in the reacting T cells. However, when the same DC are activated by various “danger” signals (microbial products or inflammatory cytokines) they then induce a T cells immune response. Autoimmunity can however arise if self-reactive T cells are not adequately eliminated or suppressed and if an activated DC then presents self-antigens to these self-reactive T cells.

DC are heterogeneous, with around five distinct types of DC in mouse lymphoid organs (Shortman and Liu, Nat Rev Immunol., 2:153-163, 2002, Vremec et al J Immunol 164:2978-2986, 2000, Henri et al J Immunol 167:741-748, 2001). In addition, a group of cells with the potential to develop into DC can be isolated, including the ‘plasmacytoid’ cells which produce class I interferons (O'Keeffe et al J Exp Med., 196:1307-1319, 2002). Although not all of these DC subtypes have been identified in humans, it is likely that a similar heterogeneity exists (Shortman and Liu, Nat Rev Immunol., 2:153-163, 2002). In mice, the DC subset characterized by high expression of CD8α⁺ DC may be especially involved in maintaining self-tolerance in its non-activated state. CD8α⁺ DC are the main DC subtype in the thymus, where they are responsible for much of the elimination of developing self-reactive T cells. CD8α⁺ DC display a number of regulatory effects in culture studies (Süss and Shortman, J Exp Med., 183:1789-1796, 1996, Kronin et al J Immunol 157:3819-3827, 1996) as well as in intact mice (Belz et al J Immunol 168:6066-6070, 2002). Thus, as well as the general picture of immature DC maintaining tolerance and activated DC producing immune responses, certain DC subtypes may have specialised roles in maintaining self-tolerance and preventing autoimmune disease.

DC, although potent in their effects, are infrequent cells and represent only a few percent of the cells in lymphoid organs. The lifespan of most DC in lymphoid organs is relatively short (Kamath et al J Immunol 165:6762-6770, 2000) although plasmacytoid pre-DC have a slower turnover (O'Keeffe et al J Exp Med 196:1307-1319, 2002). These numbers are maintained by continuous development from bone-marrow precursor cells.

There is a need to identify agents to modulate levels of DC and to develop therapeutic protocols based on altered levels of DC and more particularly of different DC types.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the determination that certain agents are capable of selectively enhancing the levels of DC or particular sub-populations thereof. The elevation of DC levels, or at least the maintenance of particular levels, assists in facilitating a state of immunological tolerance (such as when the DC are quiescent) or elevating activated DC to enhance immunity. In particular, a ligand of the tyrosine kinase receptor, Flt-3, referred to as Flt-3 ligand (Flt-3L) also known as fms-like tyrosine kinase-3 or Flk-2 (foetal liver kinase-2) is capable of selectively elevating particular sub-types of DC, such as but not limited to, plastacytoid DC and CD8⁺ DC or their equivalents in non-immune animals such as humans. It is the selective elevation of these sub-types of DC which facilitates the maintenance of a tolerogenic state in a subject. Furthermore, the elevation of activated DC assists in enhancing an immune response. The latter is important in terms of facilitating a response against a pathogenic agent. This is useful for treating pathogenic agent-induced autoimmune conditions.

The present invention provides, therefore, agents such as Flt-3L or its derivatives, homologs, chemical analogs, mimetics, chemical functional equivalents or an agonist of Flt-3L/Flt-3L receptor agonists which are useful in reducing the incidence of autoimmune pathologies and for improving the effectiveness of tolerogenic vaccines.

The agents and methods of the present invention enable prevention, or at least delay onset of, an autoimmune disease as well as enhancing the immune response against cancers and pathological agents including viruses.

Accordingly, the present invention provides a method for preventing onset of an autoimmune disease in a subject said method comprising administering to said subject an effective amount of an agent which selectively increases the levels of DC or one or more sub-types thereof.

More particularly, the present invention contemplates a method for preventing onset of an autoimmune disease such as but not limited to Type 1 diabetes (autoimmune diabetes) in a subject said method comprising administering to said subject an effective amount of Flt-3L or a derivative, homolog, chemical analog, mimetic, chemical functional equivalent or Flt-3-Flt-3L receptor agonist thereof for a time and under conditions sufficient to elevate levels of tolerance-generating or quiescent DC.

In another embodiment of the present invention contemplates modulating the degree of tolerogenicity in a subject, said method comprising administering to said subject a tolerogenic state-enhancing or maintaining effective amount of Flt-3L or a derivative, homolog, chemical analog, mimetic, chemical functional equivalent or Flt-3-Flt-3L receptor agonist.

The present invention further contemplates enhancing an immune response against cancer cells or pathogenic organisms and viruses. The aspect of the present invention permits the treatment of an autoimmune disease which is induced by a pathogenic agent. One non-limiting example is viral-induced autoimmune diabetes.

A general enhancing of the immune system is achieved by elevating levels of activated DC such as from the group comprising plastacytoid DC and CD8⁺ DC or their equivalents in non-murine animal, such as humans.

### BRIEF DESCRIPTION OF THE FIGURES

FIG. 1 is a graphical representation of flow cytometric analysis of the relative levels of DC subtypes in the spleen of NOD mice compared to NOR mice, and the changes resulting from FL treatment. The upper diagram shows the segregation of the enriched DC preparations into pDC and cDC groups (boxed, with percentages shown). The middle diagram shows the further subdivision of the CD11c^{hi} CD45RA⁻ cDC into the three subtypes (boxed, with percentages shown). The lower diagram the effects of FL treatment on these cDC subtypes. Absolute levels of each DC subtype per spleen are given in Table 2. The results shown are for mice 55 days of age. FL treated mice were analysed 1 day after the 10 day treatment. Very similar relative DC subtype levels and FL effects were obtained with mice at 110 days of age, immediately before diabetes incidence begins.

FIG. 2 is a graphical representation showing the production of IL-12 by CD8⁺ cDC from NOD or C57BL/6 mice. The CD8⁺ cDC were purified and sorted from pooled spleens, then cultured overnight with an optimal mix of cytokines and CpG as a microbial stimulus.

Stimulation with heat-killed Staphylococcus aureus gave similar results.

FIG. 3 is a graphical representation of flow cytometric analysis of the relative levels of cDC subtypes in the spleens of NOD, C57BL/6 and NOD.B6-Chr4 congenic mice. The CD11c^{hi}CD45RA⁻ cDC group was gated and subdivided as in FIG. 1. The CD4⁻8⁺ cDC subtype is boxed and its percentage of all cDC given. The absolute DC subtype levels are given in Table 2. The results shown are for mice at 55 days of age. Very similar relative DC subtype distribution was seen in all strains at 110 days of age.

FIG. 4 is a graphical representation of the cumulative diabetes incidence in female NOD mice (n=24 per group) treated for 10 days with hFL beginning at 50 days of age. Control mice were injected with the carrier medium alone. A parallel treatment of NOR mice showed no incidence of diabetes, whether FL treated or carrier alone injected.

FIG. 5 is a graphical representation of the cumulative diabetes incidence in female NOD mice treated at various ages with mFL. The control group was incubated with carrier medium alone. The upper graphs (n=16 per group) present the results from mice given a single 10 day treatment beginning at 20 days, at 50 days or at 100 days of age. The lower graph (n=18 per group) are from mice given three successive 10 day treatments beginning at 20 days, then at 50 days, then at 100 days of age.

### DETAILED DESCRIPTION OF THE PREFERRED EMBODIMENTS

The terminology used herein is for the purpose of treatment and describing particular embodiments of the subject invention only and is not intended to be limiting.

The present invention relates generally to methods of prophylaxis and agents useful for same. In particular, the present invention contemplates a method for preventing onset of an autoimmune condition, disorder or disease by the administration of an agent which selectively enhances the levels of at least DC, particularly certain DC sub-types and most particularly plastacytoid DC or CD8⁺ DC or their non-murine equivalents such as in humans. This aspect extends to a method for enhancing an immune response against cancer cells or pathogenic agents or treating an autoimmune condition by attacking a pathogenic agent inducing the autoimmune condition. One example of such a condition is viral-induced diabetes. Reference to “CD8⁺ DC” includes murine, human and non-murine equivalents of CD8⁺ DC. In a particularly preferred embodiment, the agent is Flt-3L or a derivative, homolog, chemical analog, mimetic, chemical functional equivalent and/or an Flt-3-Flt-3L receptor agonist.

Accordingly, one aspect of the present invention provides a method for preventing onset of an autoimmune disease in a subject said method comprising administering to said subject an effective amount of an agent which selectively increases the levels of DC or one or more sub-types thereof.

More particularly, the present invention contemplates a method for preventing onset of an autoimmune disease in a subject said method comprising administering to said subject an effective amount of Flt-3L or a derivative, homolog, chemical analog, mimetic, chemical functional equivalent or Flt-3-Flt-3L receptor agonist thereof for a time and under conditions sufficient to elevate levels of tolerance generating or quiescent or activated DC.

The Flt-3L or its homolog may be from the same species to which it is administered (i.e. homologous Flt-3L) or it may be from a different species (heterologous Flt-3L). An Flt-3L (or its homolog) is contemplated from humans, non-human primates, livestock animals, laboratory test animals, companion animals, captured wild animals and avian species. Examples of these types of animals are defined further below.

Preferably, the DC sub-type is a plastacytoid DC or CD8⁺ DC or their equivalent in non-murine species such as humans is/are selectively elevated in the subjects after administration of the Flt-3L or its derivative, homolog, chemical analog, mimetic, chemical functional equivalent or Flt-3-Flt-3L receptor agonist.

The present invention extends to enhancing a tolerogenic state, enhancing the effectiveness of a vaccination regime and/or modulating immune responsiveness between tolerance and immunity. These conditions are encompassed by the term “modulation” tolerance or maintaining or enhancing a tolerogenic state in a subject.

Accordingly, another aspect of the present invention contemplates modulating the degree of tolerogenicity in a subject including maintaining a state of tolerance in a subject, said method comprising administering to said subject a tolerogenic state-enhancing or -maintaining effective amount of Flt-3L or a derivative, homolog, chemical analog, mimetic, chemical functional equivalent or Flt-3-Flt-3L receptor agonist.

This aspect of the present invention extends to a method for enhancing an immune response by elevating activated DC. In a further embodiment, the present invention extends to preventing onset of a pathogenic agent-induced autoimmune disease by enhancing an immune response against the pathogen to eradicate or substantially lower same.

Generally, the administration occurs until levels of DC or sub-types thereof are elevated.

Preferably the DC sub-type is selected from plastacytoid DC or CD8⁺ DC or non-murine (eg. human) equivalents.

The present invention is generally applicable to preventing onset of an autoimmune disease, such as but not limited to Type 1 diabetes and pathogenic agent (eg. virus)-induced diabetes. This onset may be early onset or late onset. Conveniently, the treatment is appropriate for subjects who are genetically pre-disposed to an autoimmune disease or who are prone to certain autoimmune disease due to aberrations in the renin-angiotensin system such as leading to atherosclerosis, cardiac disease, obesity and/or infection.

As used in the subject specification, the singular forms “a”, “an” and “the” include plural aspects unless the context clearly dictates otherwise. Thus, for example, reference to an “agent” includes a single agent, as well as two or more agents, reference to an “Flt-3L” includes a single Flt-3L, as well as two or more Flt-3L-like molecules, and so forth.

The present invention extends, therefore, to administering Flt-3L or its derivative, homolog, chemical analog, mimetic, chemical functional equivalent, Flt-3-Flt-3L receptor agonist alone or in combination with other molecules such as Toll-like receptor ligands, tolerogenic vaccine and/or one or more other cytokines.

Although the administration of Flt-3L or its derivative, homolog, chemical analog, mimetic chemical functional equivalent or Flt-3-Flt-3L receptor agonist alone or in combination with other molecules such as Toll-like receptor ligands a tolerogenic vaccine and/or one or more other cytokines is preferred, the present invention extends to genetic means to elevate levels of Flt-3L or Flt-3L-like molecules or to down-regulate expression of genetic systems which inhibit production of Flt-3L or Flt-3L-like molecules. Genetic means include sense and anti-sense deoxyribonucleotides or ribodeoxyribonucleotides, interfering RNA, RNAi, short interfering RNA and ribozymes.

The term “subject” includes inter alia an individual, patient, target, host or recipient regardless of whether the subject is a human or non-human animal including avian species. The term “subject”, therefore, includes a human, non-human primate (eg. gorilla, marmoset, African Green Monkey), livestock animal (eg. sheep, cow, pig, horse, donkey, goat), laboratory test animal (eg. rat, mouse, rabbit, guinea pig, hamster), companion animal (eg. dog, cat), captive wild animal (eg. fox, deer, game animals) and avian species including poultry birds (eg. chickens, ducks, geese, turkeys).

The preferred subject, however, is a human. However, insofar as the present invention extends to an animal model, the subject may be a mouse, rat, pig, sheep, non-human primate or other non-human animal.

The “agent” may also be referred to as therapeutic molecule, prophylactic molecule, compound, active, or active ingredient. The terms “agent”, “therapeutic molecule”, “prophylactic molecules”, “compound”, “active” and “active ingredient” includes Flt-3L or a derivative, homolog, chemical analog, mimetic, chemical functional equivalent or Flt-3-Flt-3L receptor agonist. Furthermore, the agent, therapeutic molecule, prophylactic molecule, compound, active or active ingredient may also be a single type of molecule or multiple (eg. two or more) types of molecules such as Flt-3L and one or more of a derivative, homolog, chemical analog, mimetic, chemical functional equivalent, Flt-3-Flt-3L receptor agonist and/or another cytokine such as a Toll-like receptor ligand. In a further embodiment, the Flt-3L or its derivative, homolog, chemical analog, mimetic, chemical function equivalent or Flt-3-Flt-3L receptor agonist may be fused to another molecule such as cytokine or Toll-like receptor ligand or other DC-activity agent. By “fusion” means chemical bond formulation between two or more molecules or an association together such as in a complex or aggregate.

Insofar as multiple agents are administered, these may be provided simultaneously or sequentially. By sequentially means within nanosecond, seconds, minutes, hours, days or weeks or other time intervals. “Simultaneously” includes administration of fusion molecules.

The amount of therapeutic compound administered is referred to as the “effective amount”.

The term “effective amount” of an agent means a sufficient amount of the agent to provide the desired therapeutic or physiological effect when administered under appropriate or sufficient conditions and amounts. Thus, an “effective amount” of an agent includes a sufficient amount of the agent to elevate levels of DC on a sub-type thereof such as plastacytoid DC or CD8⁺ DC or their non-murine (eg. human) equivalents. Single or multiple doses may be administered. Undesirable effects, eg. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. The term “practitioner” would include a human medical practicione, veterinarian or medical scientist.

Effective amounts may be measured from ng/kg body weight to g/kg body weight per minute, hour, day, week or month.

The agents of the present invention may be chemical or proteinaceous molecules.

In relation to proteinaceous molecules, including peptides, polypeptide and proteins, without distinction, the terms mutant, part, derivative, homolog, analog or mimetic are meant to encompass alternative forms of Flt-3L or its homologs which interact with the Flt-3 receptor to enhance levels of DC or sub-types thereof.

Mutant forms may be naturally occurring or artificially generated variants of Flt-3L or its homologs comprising one or more amino acid substitutions, deletions or additions.

Mutants may be induced by mutagenesis or other chemical methods or generated recombinantly or synthetically. Alanine scanning is a useful technique for identifying important amino acids (Wells, Methods Enzymol., 202: 2699-2705, 1991). In this technique, an amino acid residue is replaced by Ala and its effect on the peptide's activity is determined. Each of the amino acid residues of the peptide is analyzed in this manner to determine the important regions of the polypeptide. Mutants are tested for their ability to bind to Flt-3L receptor and for other qualities such as ability to be phosphorylated, longevity, binding affinity, dissociation rate, ability to cross membranes or ability to enhance levels of DC or sub-types thereof.

Parts of the instant agents encompass Flt-3L receptor binding portions of the full-length Flt-3L. Parts are at least 10, preferably at least 20 and more preferably at least 30 contiguous amino acids, which exhibit the requisite activity. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of an amino acid sequence of the invention with proteinases such as endoLys-C, endoArg-C, endoGlu-C and staphylococcus V8-protease. The digested fragments can be purified by, for example, high performance liquid chromatographic (HPLC) techniques. Any such fragment, irrespective of its means of generation, is to be understood as being encompassed by the term “derivative” as used herein.

Thus derivatives, or the singular derivative, encompass parts, mutants, homologs, fragments, analogues as well as hybrid or fusion molecules and glycosylation variants. Derivatives also include molecules having a percent amino acid sequence identity over a window of comparison after optimal alignment. Preferably, the percentage similarity between a particular sequence and a reference sequence is at least about 60% or at least about 70% or at least about 80% or at least about 90% or at least about 95% or above such as at least about 96%, 97%, 98%, 99% or greater. Preferably, the percentage similarity between species, functional or structural homologs of the instant agents is at least about 60% or at least about 70% or at least about 80% or at least about 90% or at least about 95% or above such as at least about 96%, 97%, 98%, 99% or greater. Percentage similarities or identities between 60% and 100% are also contemplated such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs. This term also does not exclude modifications of the polypeptide, for example, glycosylations, acetylations, phosphorylations and the like. Included within the definition are, for example, polypeptides containing one or more analogs of an amino acid (including, for example, unnatural amino acids such as those given in Table 1) or polypeptides with substituted linkages. Such polypeptides may need to be able to enter the cell.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 1.

TABLE 1
Codes for non-conventional amino acids

| Non-conventional | |
|---|---|
| amino acid | Code |
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-Nmethylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N _{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Mimetics are another useful group of compounds. The term is intended to refer to a substance which has some chemical similarity to the molecule it mimics, specifically Flt-3L or a homolog thereof but which antagonizes or agonizes (mimics) its interaction with the Flt-3L receptor. A peptide mimetic may be a peptide-containing molecule that mimics elements of protein secondary structure (Johnson et al Peptide Turn Mimetics in Biotechnology and Pharmacy, Pezzuto et al Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule. Peptide or non-peptide mimetics of Flt-3L may be useful as an agent which enhances the levels of DC or sub-types thereof.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modeled. This can be especially useful where the ligand and/or binding partner change conformation on binding, allowing the model to take account of this in the design of the mimetic. Modelling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, eg. Hodgson (Bio/Technology 9: 19-21, 1991). In one approach, one first determines the three-dimensional structure of a protein of interest by x-ray crystallography, by computer modelling or most typically, by a combination of approaches. Useful information regarding the structure of a polypeptide may also be gained by modelling based on the structure of homologous proteins. An example of rational drug design is the development of HIV protease inhibitors (Erickson et al Science 249: 527-533, 1990).

One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, the formation of complexes between a target or fragment and the agent being tested, or examine the degree to which the formation of a complex between a target or fragment and a known ligand is aided or interfered with by the agent being tested.

The screening procedure includes assaying (i) for the presence of a complex between the drug and the target, or (ii) an alteration in the expression levels of nucleic acid molecules encoding the target. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target antagonists or agonists.

Purified target can be coated directly onto plates for use in the aforementioned drug screening techniques. However, non-neutralizing antibodies to the target may also be used to immobilize the target on the solid phase. The target may alternatively be expressed as a fusion protein with a tag conveniently chosen to facilite binding and identification.

Accordingly the present invention also provides an agent for modulating the levels of DC and/or a tolerogenic state which mimic Flt-3L or its homologs or which agonize Flt-3L interaction with its receptor.

Such agents may be identified and isolated as a result of screening programs or they may be developed based on the 1-D, 2-D or 3-D structure of Flt-3L, its receptor or its homologs.

Following identification of a suitable agent, it may be manufactured and/or used in a preparation, i.e. in the manufacture or formulation or a composition such as a medicament, pharmaceutical composition or drug. These may be administered to individuals in a method of treatment or prophylaxis. Alternatively, they may be incorporated into a patch or slow release capsule or implant.

The terms “compound”, “active agent”, “pharmacologically active agent”, “medicament”, “active” and “drug” are used interchangeably herein to refer to a chemical compound that induces a desired pharmacological and/or physiological effect. As discussed above, the active agents may be bound together, fused together and/or presented by an aggregate or complex. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The term “compound” is not to be construed as a chemical compound only but extends to peptides, polypeptides and proteins as well as genetic molecules such as RNA, DNA and chemical analogs thereof.

Thus, the present invention extends, therefore, to a pharmaceutical composition, medicament, drug or other composition including a patch or slow release formulation comprising an agent of the present invention which modulates levels of DC or sub-types thereof and maintains or enhances a state of tolerance or enhances an immune response in a subject.

Furthermore, the present invention contemplates a method of making a pharmaceutical composition comprising admixing a compound of the instant invention with a pharmaceutically acceptable excipient, vehicle or carrier, and optionally other ingredients. Where multiple compositions are provided, then such compositions may be given simultaneously or sequentially. Sequential administration includes administration within nanoseconds, seconds, minutes, hours or days. Preferably, within seconds or minutes.

In relation to genetic molecules, the terms mutant, part, derivative, homolog, analog or mimetic have, mutatis mutandis, analogous meanings to the meanings ascribed to these forms in relation to proteinaceous molecules. In all cases, variant forms are tested for their ability to function as proposed herein using techniques which are set forth herein or which are selected from techniques which are currently well known in the art.

When in nucleic acid form, a derivative comprises a sequence of nucleotides having at least 60% identity to the parent molecule or portion thereof. A “portion” of a nucleic acid molecule is defined as having a minimal size of at least about 10 nucleotides or preferably about 13 nucleotides or more preferably at least about 20 nucleotides and may have a minimal size of at least about 35 nucleotides. This definition includes all sizes in the range of 10-35 nucleotides including 10, 11, 12, 13, 14, 15, 16, 17, 18, 19, 20, 21, 22, 23, 24, 25, 26, 27, 28, 29, 30, 31, 32, 33, 34 or 35 nucleotides as well as greater than 35 nucleotides including 50, 100, 300, 500, 600 nucleotides or nucleic acid molecules having any number of nucleotides within these values. Having at least about 60% identity means, having optimal alignment, a nucleic acid molecule comprises at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identity with a reference Flt-3L-encoding molecule.

Alternatively, or in addition, the derivative or homolog nucleic acid molecule is defined on the basis of its ability to hybridize to a reference sequence (or a complementary form thereof) under low stringency conditions.

The terms “similarity” or “identity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and amino acid sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Preferably, the percentage similarity between a particular sequence and a reference amino acid sequence is at least about 60% or at least about 70% or at least about 80% or at least about 90% or at least about 95% or above such as at least about 96%, 97%, 98%, 99% or greater. Percentage similarities between 60% and 100% are also contemplated such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, J. Mol. Biol. 5: 109, 1962). However, the Tₘ of a duplex nucleic acid molecule decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Reference to a nucleic acid molecule which modulates the expression of Flt-3L-encoding DNA encompasses genetic agents such as DNA (genomic, cDNA), RNA (sense RNAs, antisense RNAs, mRNAs, tRNAs, rRNAs, small interfering RNAs (SiRNAs), micro RNAs (miRNAs), small nucleolar RNAs (SnoRNAs), small nuclear (SnRNAs)) ribozymes, aptamers, DNAzymes or other ribonuclease-type complexes. Other nucleic acid molecules will comprise promoters or enhancers or other regulatory regions which modulate transcription.

Accordingly, the present invention extends to a genetic approach for modulating a tolerogenic state in a subject using nucleic acid constructs which modulate the expression of Flt-3L-encoding DNA or RNA.

In one example, nucleic acid molecules which encode Flt-3L are used to elevate levels of the Flt-3L. Alternatively, the nucleic acid molecules may induce temporary or permanent gene silencing of an inhibitor of Flt-L3.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Antisense polynucleotide sequences, for example, are useful in silencing transcripts. Furthermore, polynucleotide vectors containing all or a portion of an Flt-3L inhibitor-encoding nucleic acid molecule may be placed under the control of a promoter in an antisense orientation and introduced into a cell. Expression of such an antisense construct within a cell will interfere with target transcription and/or translation. Furthermore, co-suppression and mechanisms to induce RNAi or siRNA may also be employed. Alternatively, antisense or sense molecules may be directly administered. In this latter embodiment, the antisense or sense molecules may be formulated in a composition and then administered by any number of means to target cells.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development 7: 187-195, 1997). Such compounds are injected into embryos and the effect of interference with mRNA is observed.

In one embodiment, the present invention employs compounds such as oligonucleotides and similar species for use in modulating the function or effect of nucleic acid molecules encoding a Flt-3L-inhibiting molecule, i.e. the oligonucleotides induce transcriptional or post-transcriptional gene silencing. This is accomplished by providing oligonucleotides which specifically hybridize with one or more nucleic acid molecules encoding the endogenous ligands. The oligonucleotides may be provided directly to a cell or generated within the cell. As used herein, the terms “target nucleic acid” and “nucleic acid molecule encoding an inhibitor” have been used for convenience to encompass DNA encoding the inhibitor, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may, therefore, fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

In another embodiment of the present invention, an agent is identified which promotes Flt-3L interaction with its receptor to enhance the effects of Flt-3L.

The instant methods of the present invention find application in the prophylaxis of a wide range of conditions associated with an aberrant immune system. In a particularly contemplated aspect, the present methods are useful to prevent onset of an autoimmune disease, to maintain a tolerogenic state and/or enhance tolerogenic vaccine regimes such as against cancer or a pathological agent.

Autoimmune diseases contemplated herein include Active Chronic Hepatitis, Addison's Disease, Anti-phospholipid Syndrome, Atopic Allergy, Autoimmune Atrophic Gastritis, Achlorhydra Autoimmune, Celiac Disease, Crohns Disease, Cushings Syndrome, Dermatomyositis, Type I Diabetes, Discoid Lupus, Erythematosis, Goodpasture's Syndrome, Grave's Disease, Hashimoto's Thyroiditis, Idiopathic Adrenal Atrophy, Idiopathic Thrombocytopenia, Insulin-dependent Diabetes, Lambert-Eaton Syndrome, Lupoid Hepatitis, Lymphopenia, Mixed Connective Tissue Disease, Multiple Sclerosis, Pemphigoid, Pemphigus Vulgaris, Pernicious Anema, Phacogenic Uveitis, Polyarteritis Nodosa, Polyglandular Auto. Syndromes, Primary Biliary Cirrhosis, Primary Sclerosing Cholangitis, Psoriasis, Raynauds, Reiter's Syndrome, Relapsing Polychondritis, Rheumatoid Arthritis, Schmidt's Syndrome, Scleroderma-CREST, Sjogren's Syndrome, Sympathetic Ophthalmia, Systemic Lupus Erythematosis, Takayasu's Arteritis, Temporal Arteritis, Thyrotoxicosis, Type B Insulin Resistance, Ulcerative Colitis and Wegener's Granulomatosis.

One particularly important disease is autoimmune diabetes (or Type 1 diabetes). This disease also includes pathogenic agent-induced diabetes such as viral-induced diabetes.

Cancers contemplated herein include without being limited to, ABLI protooncogene, AIDS Related Cancers, Acoustic Neuroma, Acute Lymphocytic Leukaemia, Acute Myeloid Leukaemia, Adenocystic carcinoma, Adrenocortical Cancer, Agnogenic myeloid metaplasia, Alopecia, Alveolar soft-part sarcoma, Anal cancer, Angiosarcoma, Aplastic Anaemia, Astrocytoma, Ataxia-telangiectasia, Basal Cell Carcinoma (Skin), Bladder Cancer, Bone Cancers, Bowel cancer, Brain Stem Glioma, Brain and CNS Tumours, Breast Cancer, CNS tumours, Carcinoid Tumours, Cervical Cancer, Childhood Brain Tumours, Childhood Cancer, Childhood Leukaemia, Childhood Soft Tissue Sarcoma, Chondrosarcoma, Choriocarcinoma, Chronic Lymphocytic Leukaemia, Chronic Myeloid Leukaemia, Colorectal Cancers, Cutaneous T-Cell Lymphoma, Dennatofibrosarcoma-protuberans, Desmoplastic-Small-Round-Cell-Tumour, Ductal Carcinoma, Endocrine Cancers, Endometrial Cancer, Ependymoma, Esophageal Cancer, Ewing's Sarcoma, Extra-Hepatic Bile Duct Cancer, Eye Cancer, Eye: Melanoma, Retinoblastoma, Fallopian Tube cancer, Fanconi Anaemia, Fibrosarcoma, Gall Bladder Cancer, Gastric Cancer, Gastrointestinal Cancers, Gastrointestinal-Carcinoid-Tumour, Genitourinary Cancers, Germ Cell Tumours, Gestational-Trophoblastic-Disease, Glioma, Gynaecological Cancers, Haematological Malignancies, Hairy Cell Leukaemia, Head and Neck Cancer, Hepatocellular Cancer, Hereditary Breast Cancer, Histiocytosis, Hodgkin's Disease, Human Papillomavirus, Hydatidiform mole, Hypercalcemia, Hypopharynx Cancer, IntraOcular Melanoma, Islet cell cancer, Kaposi's sarcoma, Kidney Cancer, Langerhan's-Cell-Histiocytosis, Laryngeal Cancer, Leiomyosarcoma, Leukaemia, Li-Fraumeni Syndrome, Lip Cancer, Liposarcoma, Liver Cancer, Lung Cancer, Lymphedema, Lymphoma, Hodgkin's Lymphoma, Non-Hodgkin's Lymphoma, Male Breast Cancer, Malignant-Rhabdoid-Tumour-of-Kidney, Medulloblastoma, Melanoma, Merkel Cell Cancer, Mesothelioma, Metastatic Cancer, Mouth Cancer, Multiple Endocrine Neoplasia, Mycosis Fungoides, Myelodysplastic Syndromes, Myeloma, Myeloproliferative Disorders, Nasal Cancer, Nasopharyngeal Cancer, Nephroblastoma, Neuroblastoma, Neurofibromatosis, Nijmegen Breakage Syndrome, Non-Melanoma Skin Cancer, Non-Small-Cell-Lung-Cancer-(NSCLC), Ocular Cancers, Oesophageal Cancer, Oral cavity Cancer, Oropharynx Cancer, Osteosarcoma, Ostomy Ovarian Cancer, Pancreas Cancer, Paranasal Cancer, Parathyroid Cancer, Parotid Gland Cancer, Penile Cancer, Peripheral-Neuroectodermal-Tumours, Pituitary Cancer, Polycythemia vera, Prostate Cancer, Rare-cancers-and-associated-disorders, Renal Cell Carcinoma, Retinoblastoma, Rhabdomyosarcoma, Rothmund-Thomson Syndrome, Salivary Gland Cancer, Sarcoma, Schwannoma, Sezary syndrome, Skin Cancer, Small Cell Lung Cancer (SCLC), Small Intestine Cancer, Soft Tissue Sarcoma, Spinal Cord Tumours, Squamous-Cell-Carcinoma-(skin), Stomach Cancer, Synovial sarcoma, Testicular Cancer, Thymus Cancer, Thyroid Cancer, Transitional-Cell-Cancer-(bladder), Transitional-Cell-Cancer-(renal-pelvis-/-ureter), Trophoblastic Cancer, Urethral Cancer, Urinary System Cancer, Uroplakins, Uterine sarcoma, Uterus Cancer, Vaginal Cancer, Vulva Cancer, Waldenstrom's-Macroglobulinemia, Wilms' Tumour.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. However, preferably, the invention is used to prevent an autoimmune disease from developing. Generally, such a condition or disorder involves an autoimmune disease or a condition such as cancer where the aim is to improve effectuous of a vaccine against the cancer. A “patient” as used herein refers to an animal, preferably a mammal and more preferably human who can benefit from the pharmaceutical formulations and methods of the present invention. There is no limitation on the type of animal that could benefit from the presently described pharmaceutical formulations and methods. A patient regardless of whether a human or non-human animal may be referred to as an individual, subject, animal, host, target or recipient. The compounds and methods of the present invention have applications in human medicine, veterinary medicine as well as in general, domestic or wild animal husbandry. For convenience, an “animal” includes an avian species such as a poultry bird, an aviary bird or game bird.

The preferred animals are humans or other primates, livestock animals, laboratory test animals, companion animals or captive wild animals.

The present invention provides, therefore, a composition such as a pharmaceutical composition comprising Flt-3L or a derivative, homolog, chemical analog, mimetic, chemical functional equivalent or a Flt-3-Flt-3L receptor agonist and one or more pharmaceutically acceptable carriers, excipients or diluents.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emusifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration.

If the polynucleotide encodes a sense or antisense polynucleotide or a ribozyme or DNAzyme, expression will produce the sense or antisense polynucleotide or ribozyme or DNAzyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Agents are formulated in pharmaceutical compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. topical, intravenous, oral, intrathecal, epineural or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may dissolved in a pharmaceutical carrier and administered as either a solution of a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering these agents directly, they could be produced in the target cell, e.g. in a viral vector such as described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

The present invention is further described by the following non-limiting Examples.

### EXAMPLE 1

### Mice

All mice were produced under specific pathogen-free conditions. The NOD/Lt females used typically have a cumulative diabetes incidence of 70-80% by 300 days in our facility. The control NOR/Lt females (Prochazka et al Diabetes 41:98-106, 1992) and C57BL/6J females do not develop diabetes. To generate the NOD congenic strains, C57BL/6 females were crossed with NOD males. (NODxB6)F1 females were then backcrossed to NOD males to generate backcross 1 generation. 10 subsequent backcrosses were then performed using the appropriate backcross progeny based on genotyping results and using NOD males or females to ensure that the Y chromosome and mitochondrial genome were NOD-derived. Genotyping results were based on DNA samples extracted from tail biopsies by standard methods and typed with polymorphic markers for Chr4, as well as a ˜10cM genome wide scan including markers flanking previously described diabetes susceptibility loci (Serreze et al Curr Dir Autoimmune 4:31-67, 2001). All marker positions and approximate cM distances from the top of Chr4 were obtained from the Mouse Genome Database (www.informatics.jax.org/).

### EXAMPLE 2

### FL Treatment

Mice were injected subcutaneously once a day for 10 successive days with 10 μg FL (either human or mouse) in 0.1 ml phosphate buffered saline (PBS) containing mouse serum albumin (MSA) 1 μg/ml, as previously described (Maraskovsky et al J Exp Med 184:1953-1962, 1996; O'Keefe et al Blood 99:2122-2130, 2002). Control mice were injected with the PBS-MSA carrier solution only.

### EXAMPLE 3

### Diabetes Assessment

Once a week from 80 days of age the urine of mice was tested for glucose using BM-Test Glycemic™ strips. Mice that were positive were then checked for blood glucose levels in a retro-orbital venous blood sample. Mice were considered diabetic if blood glucose was above 11 mM on two successive days. Diabetic mice were then killed and the pancreas removed for histological assessment.

### EXAMPLE 4

### FL

The recombinant mFL was produced by a transfected Chinese hamster ovary cell line and was purified by affinity chromatography. The recombinant hFL, produced in a mammalian cell line, was provided by Searle (St. Louis, Mo.). The functional effects and dose responses of both preparations on mouse DC populations have been previously presented (O'Keefe et al 2002 supra).

### EXAMPLE 5

### Dendritic Cell Isolation, Enrichment and Analysis

Full details of the procedures for DC isolation and flow-cytometric analyses have been previously presented (Vremec et al J Immunol 164:2978-2986, 2000; O'Keefe et al J Exp Med 196:1307-1319, 2002; Henri et al J Immunol 167:741-748, 2001). To extract all DC, spleens or thymuses were chopped and subjected to a mild collagenase digestion at room temperature, then treatment with EDTA. This procedure does not activate DC. The lightest density cells were then selected by centrifugation in a Nycodenz medium. Non-DC lineages were removed by coating cells with mAb against CD3, Thy-1, CD19, GR-1 and erythrocytes, then depleting coated cells with anti-Ig magnetic beads. This procedure caused no loss of pDC. This DC-enriched preparation was then counted and labelled with up to four fluorochrome-conjugated mAb, together with propidium iodide. It was analysed (and occasionally sorted) on a FACStar Plus instrument (Becton Dickinson, San Jose, Calif.) using FL1 for fluorescein isothiocyanate, FL2 for phycoerythrin, FL3 for Cy5 and FL4 for Alexa 594, with the FL5 channel set to exclude propidium iodide-stained dead cells and autofluorescent cells. The primary division of DC was into pDC (CD11c^{int} CD45RA^{hi}) and cDC (CD11c^{hi} CD45RA⁻). The cDC were subdivided by additional staining into CD4⁹⁻8⁻, CD4⁺8⁻ and CD4⁻8⁺ subtypes.

### EXAMPLE 6

### IL-12 Production by DC

The assay procedures were as previously described (Hochrein et al J Exp Med 192:823-833, 2000; Hochrein et al J Immunol 166:5448-5455, 2001). Purified sorted DC were cultured at 0.5×10⁶ cells/ml in modified RPMI 1640 medium containing either fixed and heat killed Staphylococcus aureus or the Toll-like receptor 9 agonist CpG and an optimal mix of cytokines (GM-CSF, IFN-γ and IL-4). The production of IL-12 p70 was determined 18 hr later by ELISA assay of the supernatants, using the mAb R29A5 as the capture antibody and C17.8 mAb as the detection antibody; note that the commonly used detection mAb R15D9 does not react with NOD IL-12 p70 and so gives a false negative reading.

### EXAMPLE 7

### DC Subset Levels in NOD Mice

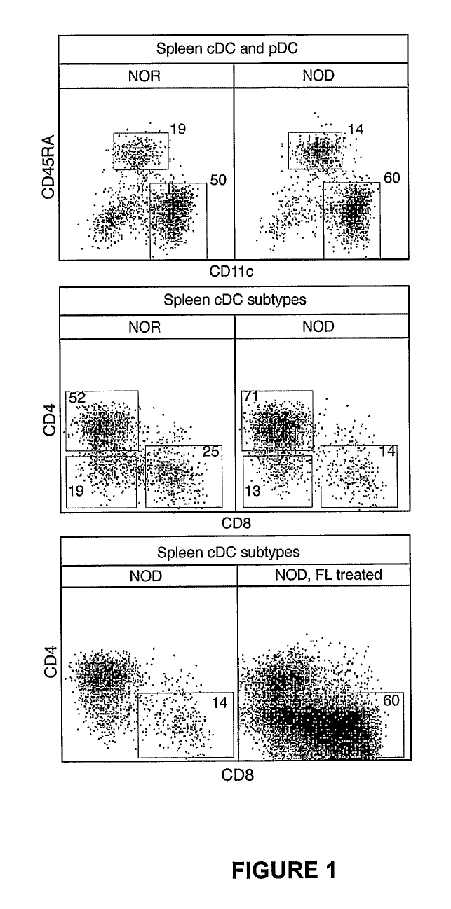

To determine if DC number or subtype balance was abnormal in NOD mice, the splenic DC populations were analysed and compared to those of the diabetes-resistant but MHC-matched and genetically similar NOR strain (Prochazka et al 1992 supra), as well as to C57BL/6 mice. NOD differed from NOR in splenic DC number and balance, both at 55 days of age (FIG. 1, Table 2), and at 110 days (just before diabetes onset). At 110 days, proportions were similar to those found at 55 days but absolute DC levels were 30% lower in both strains. NOD mice had a slightly reduced level of DC overall compared to NOR, a moderate reduction in CD4⁻8⁻ DC, but a more striking absolute and relative reduction in the CD8⁺ cDC subtype [p>0.001]. The major CD4⁺8⁻ splenic DC subtype was present at normal levels. This is in line with Prasad and Goodnow Int Immunol 14:677-684, 2002 who reported over representation of CD8⁻ DC in NOD mice and Vasquez et al Clin Exp Immunol 135:209-218, 2004 who reported a deficiency in CD8⁺ DC. In contrast, the inventors no alteration in DC levels in the thymus, where CD4⁻8⁺ DC are the major population. The DC in the NOD spleen presented similar levels of MHC II, costimulator molecules and a range of other markers to DC in NOR spleen. The C57BL/6 mice were generally similar to the NOR controls in relative DC subset distribution, the only differences noted being lower surface expression of CD11c compared to both NOD and NOR, and a higher number of DC overall (Table 2). The relative deficiency in peripheral CD8⁺ cDC in NOD mice was apparent regardless of which strain was used for comparison.

### EXAMPLE 8

### Bioactive IL-12 Production by CD8⁺ DC of NOD Mice

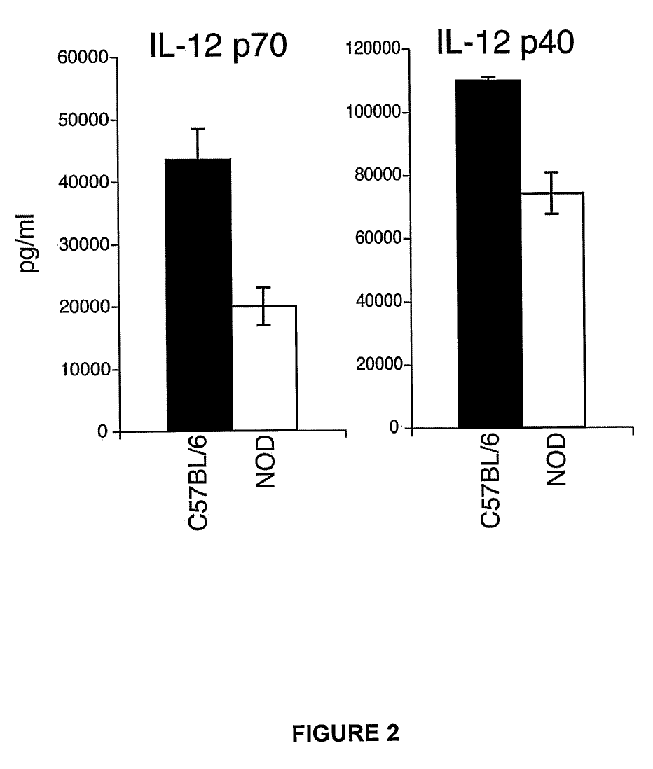

To check if the CD8⁺ cDC that remained in NOD mice were normial in function, the capacity of the cells to produce IL-12 was tested. CD8⁺ cDC normally have a much greater capacity to produce bioactive IL-12 p70 than other DC subtypes (Hochrein et al 2001 supra) and accordingly tend to induce Th1 responses. Purified and sorted CD8⁺ cDC from NOD mouse spleen were cultured using stimuli previously shown to induce maximal IL-12 production (Hochrein et al 2000 supra). The NOD CD8⁺ cDC were able to produce substantial amounts of IL-12 p40, and of the bioactive IL-12 p70, although only about half that produced by CD8⁺ cDC from C57BL/6 mice (FIG. 2). It is not clear whether this partial reduction in IL-12 production capacity would have immunological consequences in vivo.

TABLE 2
DC subtype levels in the spleen

| | Dendritic cells (×10⁶) per spleen | | | | |
|---|---|---|---|---|---|
| | | | CD4⁻8⁺ | CD4⁺8⁻ | |
| Strain | Total pDC | Total cDC | cDC | cDC | CD4⁻8⁻ cDC |
| C57BL/6 | 0.61 ± 0.27 | 2.64 ± 0.40 | 0.53 ± 0.08 | 1.32 ± 0.20 | 0.50 ± 0.07 |
| NOR | 0.54 ± 0.10 | 1.78 ± 0.27 | 0.39 ± 0.06 | 0.93 ± 0.14 | 0.34 ± 0.05 |
| NOD | 0.49 ± 0.14 | 1.53 ± 0.17 | 0.18 ± 0.02 | 1.09 ± 0.12 | 0.21 ± 0.02 |
| NOD, FL treated | 12.74 ± 3.76 | 18.58 ± 2.23 | 11.75 ± 1.91 | 2.36 ± 0.48 | 3.92 ± 1.01 |
| NOD. B6-Chr4 | 0.56 ± 0.13 | 1.71 ± 0.40 | 0.39 ± 0.09 | 0.94 ± 0.22 | 0.27 ± 0.06 |

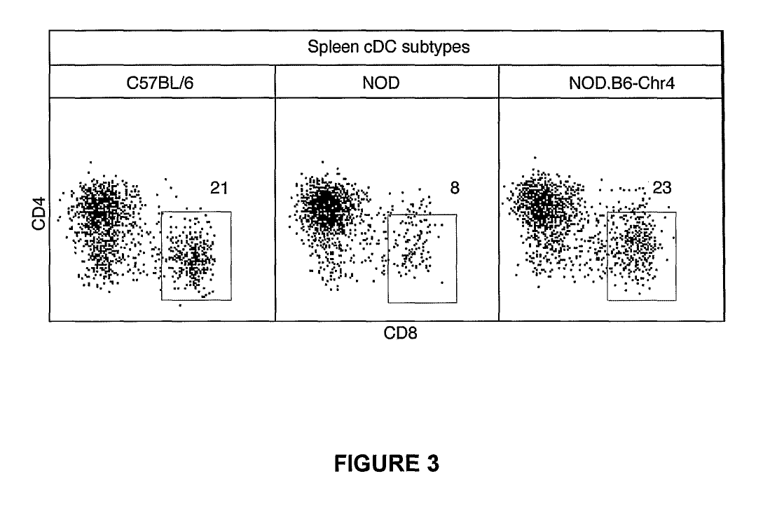

The total level of various DC subtypes in the spleens of NOD mice, compared to NOR and C57BL/6 control mice, and to congenic NOD.B6-Chr4 mice. The effect of FL treatment on the levels in NOD mice is also presented. Results were calculated from a total cell count in the DC enriched preparation combined with the relative levels in analyses such as FIGS. 1 and 3. The mice were around 55 days of age. Results represent the means±SD of 5 experiments, except for the FL treated which are the means±range of 2 experiments. All assays were on a pool of at least 3 spleens.

### EXAMPLE 9

### Genetic Factors Underlying the DC Abnormality in NOD Mice

To determine whether the genetic variation which contributes to the DC deficiencies in NOD mice also predisposes to diabetes onset, congenic mouse strains were used. A NOD congenic mouse strain was generated with a C57BL/6-derived interval on chromosome 4 (termed NOD.B6-Chr4). This interval is located distal to D4Mit31 (˜51.3cM) and encompasses all distal markers up to and including D4Mit256 (˜82.7cM). NOD.B6-Chr4 congenic female mice (n=53) were monitored for diabetes and demonstrated a marked decrease (from 72% to 32% by 300 days of age, P>0.001) in the incidence of diabetes compared to NOD female mice (n=56). The splenic DC subtype levels and balance in the NOD.B6-Chr4 congenic mice were compared to those of NOD, NOR and C57BL/6.

Substitution of the C57BL/6-derived interval on chromosome 4 increased the level of CD8⁺ cDC in the spleen [p>0.001] to the level in NOR mice (Table 2, FIG. 3). The absolute level of DC overall did not rise and so remained below that of C57BL/6 mice. There was some increase in the levels of pDC and CD4⁻8⁻ DC, but these did not reach statistical significance. The overall result was that the relative levels of CD8⁺ cDC and the normal DC subtype balance was restored (FIG. 3). No changes in the levels of T cells, B cells or other haemopoietic cells were observed. There was no change in thymic DC. The deficiency in peripheral CD8⁺ cDC is therefore determined by genes in this large interval of chromosome 4 of NOD mice, which includes known diabetes susceptibility loci.

### EXAMPLE 10

### Effect of FL Administration on DC in NOD Mice

The inventors have shown that administration of FL to C57BL/6 mice not only elevates DC levels overall but selectively elevated the CD8⁺ cDC and the pDC subtypes (Maraskovsky et al 1996 supra; O'Keefe et al 2002 supra). This was especially apparent with mouse FL (mFL) although the overall elevation in DC levels was less than with human FL (hFL). Following FL treatment, levels of MHC II and costimulator molecules were characteristic of quiescent rather than activated DC. DC levels declined back to baseline one week after treatment. Since the CD8⁺ cDC subtype deficient in NOD mice was increased by FL in C57BL/6 mice, we asked if FL treatment could rectify the NOD DC imbalance.

NOD mice were treated with 10 μg of either hFL or mFL per day for 10 days, then the DC levels and subpopulation balance in the spleens determined on day 11. Both treatments caused an increase in overall DC levels. The level of CD8⁺ cDC and pDC was markedly elevated, which became the dominant DC subtypes (FIG. 1 and Table 2). The CD4⁻8⁻ and CD4⁺8⁻ cDC subsets increased but to a smaller extent than for CD8⁺ cDC.

### EXAMPLE 11

### Reduction in NOD Mouse Diabetes Incidence Following hFL Treatment

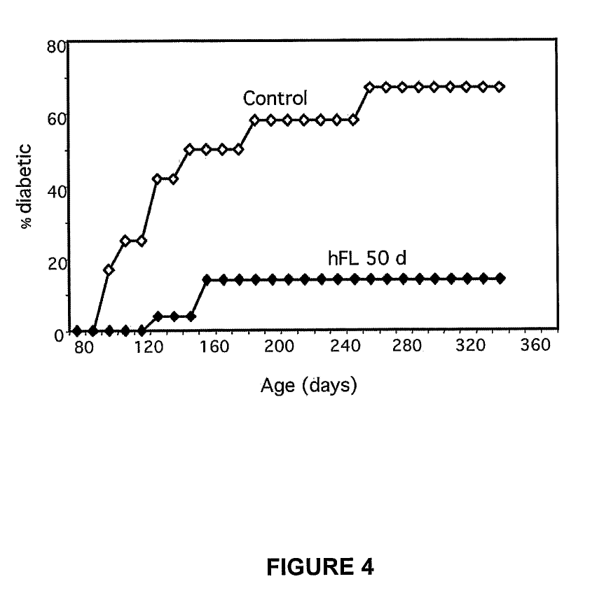

Since FL treatment transiently corrected the DC imbalance in NOD mice, and for about 5 days even made CD8⁺ DC the dominant subtype, it was determined whether such treatment would reduce or delay diabetes incidence. Female NOD mice were treated at age 50 days with either hFL or mFL and the incidence of diabetes serially monitored. The most striking effect was with hFL, which caused the greatest elevation in DC levels. The cumulative incidence of diabetes, which began around 100 days, was reduced from 68% to 14% [p>0.001](FIG. 4). Although FL treatment began at 50 days and the effect on DC levels would not have been evident beyond 70 days, there appeared to be a long-term effect to prevent diabetes development. In a parallel study, no NOR mice, whether FL-treated or untreated, became diabetic.

Sections of the pancreas of hFL-treated NOD mice, untreated NOD mice, and of control mice aged 195 days of age were examined histologically and assessed for mononuclear cell infiltration and destruction of pancreatic islets. The histology was in general accordance with the diabetes incidence results. The NOR control mice had a mean of 43 islets per 7 spaced longitudinal sections, and a mean of 57 islets per 7 sections after hFL treatment; most islets showed mild peri-islet mononuclear cell infiltration, in contrast to C57BL/6 mice which showed no infiltration. Untreated NOD mice classed as diabetic had a mean of only 9 islets per 7 sections, compared to those classed as non-diabetic which had 3 5 islets per 7 sections; in both cases there was a high level of mononuclear cell infiltration in the remaining islets and partial destruction of the beta cells. The hFL treated NOD mice which were non-diabetic (the majority) had a mean of 47 islets per 7 sections, with the islets showing a moderate level of mononuclear cell infiltration but only a low level of beta cell destruction.

### EXAMPLE 12

### Complete Prevention of Diabetes Development with Repeated Doses of mFL

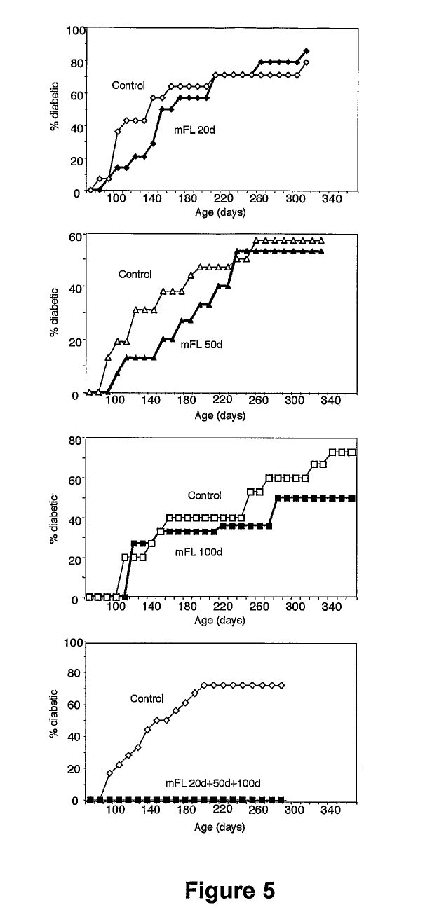

A single administration of mFL only transiently inhibited diabetes development. The effects of different times of administration of mFL were then tested. When NOD mice were given 10 days of mFL treatment beginning at age 20 days, when the autoimmune process is just beginning, there was a small but not statistically significant reduction in diabetes incidence between 100 and 150 days of age, but no difference thereafter (FIG. 5). When mFL was administered beginning at 50 days of age, there was an apparent reduction in diabetes incidence between 120-200 days, but in contrast to the results with hFL, the effect was of marginal significance and eventually the mice became diabetic (FIG. 5). When mFL was administered at 100 days, when beta-cell destruction is beginning, there was no evidence of immediate protection from diabetes development, but the data suggested a reduction in late-onset diabetes incidence, from 250-370 days (FIG. 5).

These data show that mFL treatment provided a transient window of protection from the initiation of an autoimmune process that could begin at any time over the young adult life of NOD mice, cumulating in diabetes about 100 days later. To test whether repeated administration of mFL, covering all the “windows”, might block initiation of the autoimmune process entirely. One group of NOD mice were combined with all the previous mFL treatments, by giving mFL for 10 days starting at 20 days, again at 50 days and again at 100 days of age. This repeated administration of mFL had the predicted effect of preventing diabetes development completely [p>0.001] (FIG. 5). Not a single mFL treated NOD mouse developed diabetes, even by 300 days of age, whereas the cumulative incidence in the untreated mice reached 75%.

### EXAMPLE 13

### Analysis of the DC Populations of NOD Mice

DC were isolated from the spleens of 8 wk old NOD mice (well before autoimmune destruction of the pancreatic β cells or overt diabetes develops), using the isolation and analysis techniques of Vremec 2000 supra; O'Keefe et al 2002 supra; Henri et al 2001 supra). The levels of DC were compared with those of the closely related but non-diabetic NOR mice (which in turn are similar to other normal mouse strains such as C57BL/6). Total levels of conventional DC subtypes for the spleen before and after FL treatment are shown in Tables 3 and 4.

TABLE 3
Number of plasmacytoid and conventional
DC per spleen of NOD and NOR female mice
Conventional dendritic cells per spleen (×10⁶)

| Strain | Total No. | CD8⁺4⁻ | CD8⁻4⁺ | CD4⁻8⁻ |
|---|---|---|---|---|
| NOR | 2.79 | 0.72 | 1.37 | 0.56 |
| NOD | 2.08 | 0.24 | 1.46 | 0.27 |

Plasmacytoid dendritic cells per spleen (×10⁵)

| Strain | Total No. |
|---|---|
| NOR | 3.03 |
| NOD | 2.11 |

TABLE 4
Numbers of conventional and plasmacytoid DC per spleen
of NOD mice after 10 days of in vivo FL treatment

| | | | |
|---|---|---|---|
| Total No. | CD8⁺4⁻ | CD8⁻4⁺ | CD4⁻8⁻ |

Conventional DC per spleen (×10⁶) of NOD mice after FL treatment*

| | | | |
|---|---|---|---|
| 17.0 (8×) | 10.4 (43×) | 2.7 (1.8×) | 3.2 (11.9×) |

Plasmacytoid DC per spleen (×10⁵) of NOD mice after FL treatment
15.4 (73×)
*The level of enhancement is given in parentheses.

All three previously established DC subtypes (CD4⁺8⁻, CD4⁻8⁻, CD4⁻8⁺) were present in both NOD and control NOR mice. However, there was a reduction in both the proportion and the absolute number of the CD4⁻8⁺ DC subtypes in NOD mice, and a small overall drop in the level of total DC numbers in NOD mice.

The levels of the plasmacytoid ‘pre-DC’ population in the two strains was also compared.

NOD mice had a reduced level of plasmacytoid pre-DC, as well as of the conventional CD8⁺ DC.

It is concluded that the development of autoimmunity in NOD mice was in part due to an overall reduction in the number of quiescent tolerogenic DC, or due to a reduced number of one particular DC subtype, or due to an imbalance in the ratio between different DC subtypes. A reduced number of DC (especially CD8α⁺ DC) in the thymus could lead to a less efficient central tolerance. A reduced number of CD8α⁺ DC in the periphery could lead to less effective peripheral tolerance via a number of mechanisms.

The overall level of DC in all lymphoid organs is increased markedly by administration of Flt-3L. In addition, there is a proportionally greater increase in CD8⁺ DC and plasmacytoid DC than of CD8⁻ DC. This change in ratio is especially noticeable when murine Flt-3L (mFlt-3L) was used, although mFlt-3L produced a lower increase in DC overall than did human Flt-3L (hFlt-3L).

Accordingly, the effect of Flt-3L administration on NOD mice was tested, to see if the DC levels could also be enhanced in this mouse strain, and if the DC subset imbalance could be rectified. The NOD mouse responded to Flt-3L much as had been shown for C57BL/6 mice (Table 3). There was an overall increase in DC in all lymphoid organs tested (including the thymus as well as spleen). In addition, the level of CD8⁺ DC and of plasmacytoid DC was differentially enhanced, so in the treated mice these were no longer relatively low but now relatively high compared with untreated normal mice.

### EXAMPLE 14

### Prevention of Diabetes by Flt-3L Injections

Since any deficiency in DC overall and any relative deficiency in CD8⁺ DC and plasmacytoid DC in NOD mice was overcome by Flt-3L treatment, the effect of such treatment on the incidence of diabetes in NOD mice was tested. A series of NOD mice was treated at 50 days of age with hFlt-3L (10 μg per day for 10 days) then the mice observed until 345 days of age. They were compared to carefully paired NOD mice injected only with the saline solvent. In addition, control NOR mice were subjected to the same regime, either Flt-3L or saline control treated. Urine samples were tested for ketone levels every week. If a positive urine test was obtained the serum was tested and a positive diagnosis of diabetes made if blood sugar levels were over 20 mmol/litre. Diabetic mice were killed and the pancreas taken for histological testing. Some control mice were also killed for pathology testing.

The cumulative incidence of diabetes in Flt-3L treated versus saline treated NOD mice revealed that in a total of 24 Flt-3L treated and 24 saline control NOD mice there was a very marked and long term reduction in diabetes incidence as a result of hFlt-3L treatment of NOD mice.

In the control experiments with NOR mice, no mice, either Flt-3L treated or control, became diabetic. No increased mortality or other signs of pathology or distress were seen as a result of the Flt-3L treatment.

The histological sections confirmed the autoimmune destruction of pancreatic tissue in the untreated NOD mice and all mice which became diabetic, and the marked reduction of this with Flt-3L treatment. Some insulitis (mononuclear cell invasion of the pancreas) was seen in the protected, Flt-3L treated mice, but the destruction of β-cells was markedly reduced. Similar insulitis was seen in the NOR mice, none of which became diabetic.

### EXAMPLE 15

### The Timing of Flt-3L Treatment

Flt-3L treatment might have prevented diabetes by either blocking the final effector phase of autoimmune β cell destruction, or may have acted earlier in preventing the generation of the initial autoimmune response. The fact that 10 days of treatment from day 50 prevented autoimmune diabetes which was normally manifest after 100 days of age suggested an effect on the initiation rather than the effector phase. To test this, the effects of Flt-3L treatment very early (20 days), at 50 days or at 100 days (just before the final autoimmune destruction was initiated) was compared.

Due to the lack of a hFlt-3L supply (a worldwide problem) these experiments used mFlt-3L. This had a reduced effect on DC levels (12 fold DC enhancement compared to 30 fold for hFlt-3L) and the effect on diabetes incidence proved to be more transient. However this allowed some important aspects of the timing of the effect to be studied.

Administration at 20 days to 30 days of age gave some reduction in diabetes incidence from 100 to 200 days, but after 200 days of age there was no reduction in diabetes incidence; the onset of diabetes was simply delayed. Administration of mFlt-3L at 50 days reduced the incidence of diabetes up to 230 days, and especially from 120 to 230 days, but again (and in contrast to hFlt-3L) the protection was not permanent and there was no difference in cumulative incidence after 240 days. Administration of mFlt-3L at 100 days had no effect on the initial incidence of diabetes, only a minor effect from 170-250 days, but the effect on the cumulative incidence was marked after 250 days. Thus administration of mFlt-3L at 100 days had no protective effect on those mice where autoimmune destruction was already underway, but strongly protected mice with late-onset diabetes. Overall Flt-3L treatment had its protective effect 80-150 days after administration commenced. Its effect therefore appears to be on the initiation of the autoimmune response, and not on the effector phase of p cell destruction once the autoimmune process is initiated.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Belz et al Immunol Cell Biol, 80:463-468, 2002
Belz et al J Immuno, 168:6066-6070, 2002
Erickson et al. Science 249: 527-533, 1990
Henri et al J Immunol 167:741-748, 2001
Hochrein et al J Exp Med 192:823-833, 2000
Hochrein et al J Immunol 166:5448-5455, 2001
Johnson et al Peptide Turn Mimetics in Biotechnology and Pharmacy, Pezzuto et al., Eds., Chapman and Hall, New York, 1993
Kamath et al J Immunol, 165:6762-6770, 2000
Kronin et al J Immunol., 157:3819-3827, 1996
Maraskovsky et al J Exp Med 184:1953-1962, 1996
Matzinger Annu Rev Immunol, 12:991-1045, 1994
O'Keefe et al Blood 99:2122-2130, 2002
O'Keefe et al J Exp Med 196:1307-1319, 2002
Prasad and Goodnow Int Immunol 14:677-684, 2002
Prochazka et al Diabetes 41:98-106, 1992
Serreze et al Curr Dir Autoimmune 4:31-67, 2001
Shortman and Liu, Nat Rev Immunol, 2:153-163, 2002
Steinman et al Ann N Y Acad. Sci., 987:15-25, 2003
Süss and Shortman, J Exp Med., 183:1789-1796, 1996
Vasquez et al Clin Exp Immunol 135:209-218, 2004
Vremec et al J Immunol 164:2978-2986, 2000
Wells Methods Enzymol., 202: 2699-2705, 1991

1. A method for preventing onset of an autoimmune disease in a subject said method comprising administering to said subject an effective amount of Flt-3L and/or a derivative, homolog, chemical analog, mimetic, chemical functional equivalent thereof or a Flt-3-Flt-3L receptor agonist which selectively increases the levels of DC or one or more sub-types thereof.

2. The method of claim 1 wherein the agent is Flt-3L.

3. The method of claim 1 or 2 wherein the Flt-3L and/or derivative, homolog, chemical analog, mimetic, chemical functional equivalent thereof or a Flt-3-Flt-3L receptor agonist is co-administered with a cytokine.

4. The method of claim 2 or 3 wherein the Flt-3L and/or derivative, homolog, chemical analog, mimetic, chemical functional equivalent thereof or a Flt-3-Flt-3L receptor agonist is co-administered with a Toll-like receptor ligand.

5. The method of claim 3 or 4 wherein co-administration is sequential administration.

6. The method of claim 3 or 4 wherein co-administration is simultaneous administration.

7. The method of claim 1 wherein the subject is a human, non-human primate, livestock animal, laboratory test animal, a companion animal, a captured wild animal or an avian species.

8. The method of claim 7 wherein the subject is a human.

9. The method of claim 1 wherein the Flt-3L or its homolog is derived from the same species to which it is administered.

10. The method of claim 1 wherein the Flt-3L or its homolog is derived from a different species to which it is administered.

11. The method of claim 1 wherein the autoimmune disease is Active Chronic Hepatitis, Addison's Disease, Anti-phospholipid Syndrome, Atopic Allergy, Autoimmune Atrophic Gastritis, Achlorhydra Autoimmune, Celiac Disease, Crohns Disease, Cushings Syndrome, Dermatomyositis, Type I Diabetes, Discoid Lupus, Erythematosis, Goodpasture's Syndrome, Grave's Disease, Hashimoto's Thyroiditis, Idiopathic Adrenal Atrophy, Idiopathic Thrombocytopenia, Insulin-dependent Diabetes, Lambert-Eaton Syndrome, Lupoid Hepatitis, Lymphopenia, Mixed Connective Tissue Disease, Multiple Sclerosis, Pemphigoid, Pemphigus Vulgaris, Pernicious Anema, Phacogenic Uveitis, Polyarteritis Nodosa, Polyglandular Auto. Syndromes, Primary Biliary Cirrhosis, Primary Sclerosing Cholangitis, Psoriasis, Raynauds, Reiter's Syndrome, Relapsing Polychondritis, Rheumatoid Arthritis, Schmidt's Syndrome, Scleroderma —CREST, Sjogren's Syndrome, Sympathetic Ophthalmia, Systemic Lupus Erythematosis, Takayasu's Arteritis, Temporal Arteritis, Thyrotoxicosis, Type B Insulin Resistance, Ulcerative Colitis and Wegener's Granulomatosis.

12. The method of claim 11 wherein the autoimmune disease is diabetes.

13. A method of modulating the degree of tolerogenicity in a subject, or enhancing the level of an immune response against cancer or a pathogenic agent said method comprising administering to said subject an effective amount of Flt-3L or a derivative, homolog, chemical analog, mimetic chemical functional equivalent or Flt-3-Flt-3L receptor agonist.

14. The method of claim 13 wherein the agent is Flt-3L.

15. The method of claim 13 or 14 wherein the Flt-3L and/or derivative, homolog, chemical analog, mimetic, chemical functional equivalent thereof or a Flt-3-Flt-3L receptor agonist is co-administered with a Toll-like receptor ligand.

16. The method of claim 14 or 15 wherein co-administration is sequential administration.

17. The method of claim 14 or 15 wherein co-administration is simultaneous administration.

18. The method of claim 13 wherein the subject is a human, non-human primate, livestock animal, laboratory test animal, a companion animal, a captured wild animal or an avian species.

19. The method of claim 18 wherein the subject is a human.

20. The method of claim 13 wherein the Flt-3L or its homolog is derived from the same species to which it is administered.

21. The method of claim 13 wherein the Flt-3L or its homolog is derived from a different species to which it is administered.

22. The method of claim 13 in the treatment of cancer.

23. The method of claim 22 wherein the cancer is ABLI protooncogene, AIDS Related Cancers, Acoustic Neuroma, Acute Lymphocytic Leukaemia, Acute Myeloid Leukaemia, Adenocystic carcinoma, Adrenocortical Cancer, Agnogenic myeloid metaplasia, Alopecia, Alveolar soft-part sarcoma, Anal cancer, Angiosarcoma, Aplastic Anaemia, Astrocytoma, Ataxia-telangiectasia, Basal Cell Carcinoma (Skin), Bladder Cancer, Bone Cancers, Bowel cancer, Brain Stem Glioma, Brain and CNS Tumours, Breast Cancer, CNS tumours, Carcinoid Tumours, Cervical Cancer, Childhood Brain Tumours, Childhood Cancer, Childhood Leukaemia, Childhood Soft Tissue Sarcoma, Chondrosarcoma, Choriocarcinoma, Chronic Lymphocytic Leukaemia, Chronic Myeloid Leukaemia, Colorectal Cancers, Cutaneous T-Cell Lymphoma, Dermatofibrosarcoma-protuberans, Desmoplastic-Small-Round-Cell-Tumour, Ductal Carcinoma, Endocrine Cancers, Endometrial Cancer, Ependymoma, Esophageal Cancer, Ewing's Sarcoma, Extra-Hepatic Bile Duct Cancer, Eye Cancer, Eye: Melanoma, Retinoblastoma, Fallopian Tube cancer, Fanconi Anaemia, Fibrosarcoma, Gall Bladder Cancer, Gastric Cancer, Gastrointestinal Cancers, Gastrointestinal-Carcinoid-Tumour, Genitourinary Cancers, Germ Cell Tumours, Gestational-Trophoblastic-Disease, Glioma, Gynaecological Cancers, Haematological Malignancies, Hairy Cell Leukaemia, Head and Neck Cancer, Hepatocellular Cancer, Hereditary Breast Cancer, Histiocytosis, Hodgkin's Disease, Human Papillomavirus, Hydatidiform mole, Hypercalcemia, Hypopharynx Cancer, IntraOcular Melanoma, Islet cell cancer, Kaposi's sarcoma, Kidney Cancer, Langerhan's-Cell-Histiocytosis, Laryngeal Cancer, Leiomyosarcoma, Leukaemia, Li-Fraumeni Syndrome, Lip Cancer, Liposarcoma, Liver Cancer, Lung Cancer, Lymphedema, Lymphoma, Hodgkin's Lymphoma, Non-Hodgkin's Lymphoma, Male Breast Cancer, Malignant-Rhabdoid-Tumour-of-Kidney, Medulloblastoma, Melanoma, Merkel Cell Cancer, Mesothelioma, Metastatic Cancer, Mouth Cancer, Multiple Endocrine Neoplasia, Mycosis Fungoides, Myelodysplastic Syndromes, Myeloma, Myeloproliferative Disorders, Nasal Cancer, Nasopharyngeal Cancer, Nephroblastoma, Neuroblastoma, Neurofibromatosis, Nijmegen Breakage Syndrome, Non-Melanoma Skin Cancer, Non-Small-Cell-Lung-Cancer-(NSCLC), Ocular Cancers, Oesophageal Cancer, Oral cavity Cancer, Oropharynx Cancer, Osteosarcoma, Ostomy Ovarian Cancer, Pancreas Cancer, Paranasal Cancer, Parathyroid Cancer, Parotid Gland Cancer, Penile Cancer, Peripheral-Neuroectodermal-Tumours, Pituitary Cancer, Polycythemia vera, Prostate Cancer, Rare-cancers-and-associated-disorders, Renal Cell Carcinoma, Retinoblastoma, Rhabdomyosarcoma, Rothmund-Thomson Syndrome, Salivary Gland Cancer, Sarcoma, Schwannoma, Sezary syndrome, Skin Cancer, Small Cell Lung Cancer (SCLC), Small Intestine Cancer, Soft Tissue Sarcoma, Spinal Cord Tumours, Squamous-Cell-Carcinoma-(skin), Stomach Cancer, Synovial sarcoma, Testicular Cancer, Thymus Cancer, Thyroid Cancer, Transitional-Cell-Cancer-(bladder), Transitional-Cell-Cancer-(renal-pelvis-/-ureter), Trophoblastic Cancer, Urethral Cancer, Urinary System Cancer, Uroplakins, Uterine sarcoma, Uterus Cancer, Vaginal Cancer, Vulva Cancer, Waldenstrom's-Macroglobulinemia, Wilms' Tumour.

24. The method of claim 22 in the prophylaxis of a pathogenic agent-induced autoimmune disease.

25. The method of claim 24 wherein the autoimmune disease is Active Chronic Hepatitis, Addison's Disease, Anti-phospholipid Syndrome, Atopic Allergy, Autoimmune Atrophic Gastritis, Achlorhydra Autoimmune, Celiac Disease, Crohns Disease, Cushings Syndrome, Dermatomyositis, Type I Diabetes, Discoid Lupus, Erythematosis, Goodpasture's Syndrome, Grave's Disease, Hashimoto's Thyroiditis, Idiopathic Adrenal Atrophy, Idiopathic Thrombocytopenia, Insulin-dependent Diabetes, Lambert-Eaton Syndrome, Lupoid Hepatitis, Lymphopenia, Mixed Connective Tissue Disease, Multiple Sclerosis, Pemphigoid, Pemphigus Vulgaris, Pernicious Anema, Phacogenic Uveitis, Polyarteritis Nodosa, Polyglandular Auto. Syndromes, Primary Biliary Cirrhosis, Primary Sclerosing Cholangitis, Psoriasis, Raynauds, Reiter's Syndrome, Relapsing Polychondritis, Rheumatoid Arthritis, Schmidt's Syndrome, Scleroderma-CREST, Sjogren's Syndrome, Sympathetic Ophthalmia, Systemic Lupus Erythematosis, Takayasu's Arteritis, Temporal Arteritis, Thyrotoxicosis, Type B Insulin Resistance, Ulcerative Colitis and Wegener's Granulomatosis.

26. The method of claim 25 wherein the autoimmune disease is diabetes.

27. The method of claim 26 wherein the autoimmune disease is viral-induced diabetes.
